(19) United States
(12) Patent Application Publication
(10) Pub. No.: US 2004/0260157 A1
(43) Pub. Date: Dec. 23, 2004

(54) Method for automated screening of cervical/endocervical malignant and premalignant epithelial lesions using flow cytometry with HPV DNA fluorescent in-situ hybridization ( FISH) technology

(76) Inventor: Miguel A. Montes, Klamath Falls, OR (US)

Correspondence Address: DELLETT AND WALTERS, P. O. BOX 2786, PORTLAND, OR, 97208-2786, US

(21) Appl. No.: 10/732,191

(22) Filed: Dec. 9, 2003

Related U.S. Application Data
(60) Provisional application No. 60/480,518, filed on Jun. 20, 2003.

(51) Int. Cl. A61B 5/00

(52) U.S. Cl. 600/301

(57) ABSTRACT

Systems and methods for automated screening of cervical and endocervical epithelium for malignant and premalignant lesions comprise collecting the epithelial cell sample in a transport fluid medium. Cells suspended in such medium are analyzed by flow cytometric technology. DNA fluorescent in-situ hybridization technology is also used to identify human papilloma virus (HPV) infected cells by means of flow cytometry. Different cell populations are quantitated as part of a complete cell count (CCC). CCC data is correlated with HPV DNA in-situ hybridization results. CCC data and CCC/HPV DNA in-situ hybridization correlation results are displayed both in three dimensional graphical form as well as two dimensional dot plot graphs depicting specific gated cellular populations. Populations of interest can also be sorted for subsequent visual morphologic inspection as well as for molecular diagnostic studies.

### BACKGROUND OF THE INVENTION

Human cervical epithelium can undergo premalignant morphologic changes which have been termed cervical dysplasia or cervical intraepithelial neoplasia (CIN). CIN I, CIN II, and CIN III correspond to progressively severe forms of dysplasia; mild, moderate, and severe dysplasia respectively. The most severe form of dysplasia, CIN III, represents full thickness epithelial dysplasia or carcinoma in-situ. Higher degrees of dysplasia have increased probability of progressing to invasive carcinoma of the cervix. The probability of such progression is 1% for CIN I, 5% for CIN II, and >12% for CIN III. (Ostor AG. Natural history of cervical intraepithelial neoplasia: a critical review. Int J Gynecol Pathol 12:186-192, 1993). More recently CIN II and CIN III have been termed high grade squamous intraepithelial lesions (HSIL) due to their increased potential to progress to invasive squamous cell carcinoma. (Crum CP, Cibas E S, Lee K R. Pathology of early cervical neoplasia. New York: Church Hill Livingston, 1996.

Approximately 60 years ago, Dr. George Papanicolaou developed the so called Pap smear as a method of screening women for cervical dysplasia. The test entails scraping the cervix/endocervix transformation zone with a spatula. The scraped cells are then smeared onto a slide which is immediately fixed by an alcohol based solution. The cells fixed onto the slide are stained (Pap stain) and subsequently analyzed under the microscope. Features such as nuclear size, nuclear chromasia, nuclear irregularity, and koilocytic changes allowed the examiner to identify dysplastic cells. More recently the Bethesda system of reporting has fine tuned the morphologic criteria for identifying dysplastic cervical cells using the Pap smear. (Kurman RJ, Solomon D, The Bethesda System for reporting cervical/vaginal cytologic diagnoses, 1994: Springer-Veralg New York Inc.). This gave rise to the categorization of dysplastic cells as either low grade squamous intraepithelial lesions (LSIL) or high grade squamous intraepithelial lesions (HSIL). These in turn correspond to low grade dysplasia and high grade dysplasia respectively.

The introduction of the Pap smear revolutionized cervical screening. The rate of cervical cancer has decreased by 70% reducing cervical cancer from the most prevalent female malignancy and leading cause of cancer deaths among American women. This has made the Pap smear test the most successful cancer screening test ever employed, thus having become the standard for cervical cancer screening. (DeMay (1997) Arch. Pathol. Lab. Med. 121:229-38.)

However, the Pap smear does have its limitations. These begin almost immediately as the sample is taken. First, only a small portion of the sample collected from the patient is transferred to the slide during the smearing of the spatula collecting device, contributing to the false negative results. It is estimated that as much as 80% of the sample may be discarded with the collecting device. The specimen in the spatula is immediately smeared onto a slide and sprayed with an alcohol based fixative. Both of these steps frequently produce artifacts precluding a definitive diagnosis. If the specimen is smeared too strongly onto the slide, cells will be crushed and destroyed rendering inadequate morphology for diagnosis. Also, if more than a couple of seconds elapse from the time the spray fixative is applied, the cells will display air drying artifact. This is evidenced by artificially enlarged cells with pale nuclei, which also often preclude lesional diagnosis. There is often obscuring blood and inflammation in the smear that either renders the specimen unsatisfactory or limited. The Pap smear relies on human observer interpretation. This introduces human error as well as interoperative variability as a source of error.

The above limitations have led to false negative results ranging from 5%-28%. (Naryshkin (1997) Arch. Pathol. Lab. Med. 121:270-272; Barry (1987) McGraw-Hill Encyclopedia of Science and Technology, 6^{th} edition, Vol. 4, p. 36; and Lieu (1996) J. Fam. Pract. 42:391-9). False positive results, on the other hand, may be as high as 11.6%. (Nenning et al. Anal. Cell. Pathol. 9:61-8; and Barry (1997) McGraw-Hill Encyclopedia of Science and Technology, 6^{th} edition, Vol 4, p. 36). Due to the possibly of false positive results a repeat follow-up Pap smear or immediate colposcopy is recommended subsequent to a positive result. (Slawson et al. (1992) J. Fam. Pract. 35:271-7). False negative results are not identified until either routine subsequent smears or a symptomatic generated examination. Symptomatic generated repeat testing is often the result of advanced disease such as invasive carcinoma. Medicare pays for screening paps once every three years. Therefore for at least this population it is likely that at least three years may pass before the false negative is identified. These issues make the review of a pap smear one of the most labor intensive, least paid, and most litigious specimen that is examined under the microscope.

In the last 5 to 10 years several technologies have been developed in an effort address the limitations of the Pap test. The first and most widely accepted of these has been the so called ThinPrep pap test developed by Cytec corporation several years ago. Approximately 52% of all the cervical cytology specimens examined in the United States today are by the ThinPrep pap test. It was the first of two FDA approved automated alternatives to the preparation of cervical scrape specimens. The method was revolutionary as it was the first time that a liquid based specimen transport media and monolayer slide preparation was used for cervical specimen diagnosis.

The ThinPrep pap test methodology is basically as follows. First, a cervical/endocervical specimen is obtained by either using a spatula and brush combination or a broom with brush extension device. The collecting device is then immediately submerged in a methanol based transport media and swirled a number of times, approximately 10, in order to dislodge the cells. The sample is then placed in the ThinPrep machine where the fluid is suctioned through a semi permeable membrane by means of applying negative pressure. The membrane has microscopic pores that let fluid through but prevent the cells from passing. The negative pressure is applied in pulses required to suction a defined volume of fluid. The time interval required for the suction pulse to obtain a defined fluid volume is also monitored. As more cells adhere to the membrane, resistance requires subsequently longer suction pulse intervals to obtain the defined fluid volume. Once the suction pulse time interval reaches a predetermined threshold value all suction ceases. The predetermined threshold time interval would be one that was judged to give the greatest cellularity without having cells on top of each other or a monolayer of cells. The cells on the membrane are then transferred to a glass slide with the aid of slight positive pressure in the direction opposite to the suction. The cells now adhered to the slide are stained by the Papanicolaou method and cover slipped for microscopic examination.

There are several reasons for the increased acceptance of the ThinPrep pap test in recent years. Virtually the entire sample is collected in the preservative fluid. There is a randomized, representative transfer of cells onto the glass slide. There is an even distribution of cells onto the slide minimizing obscuring material, cellular clumping, and cellular overlap. The immediate transfer of cells onto a fluid environment eliminates mechanical and air drying artifact rendering improved morphologic detail for diagnosis. The result of this has been biopsy confirmed increased detection of HSIL by 103%, increased detection of LSIL by 72%, and a decreased ASCUS (atypical squamous cells of undetermined significance) rate by 5%. (Kabawat SE, et al., Arch Pathol Lab Med. 1999 Vol 123:817-821). Finally, Cytec Corp. has done an excellent job marketing this product.

As with the Pap smear test there are certain limitations with the ThinPrep pap test that make it less than ideal. As with the Pap smear test, this is a labor intensive method of slide preparation followed by human microscopic examination. Human error and variation of interpretation is not eliminated. The ability to judge cells by the company they keep is lost due to the so called cellular randomization of the specimen. This can be particularly problematic when trying to identify populations of atypical immature squamous metaplastic like cells that may actually be HSIL cells or associated with such. (Montes M A, Cibas ES, Lee K, DiNisco S. Cytologic Characteristics of Abnormal Cells in Prior “Normal” Cervical Vaginal Papanicolaou Smears From Women With a High Grade Squamous Intraepithelial Lesions. Cancer Cytopathology 87:56-59, 1999. Yearbook of Pathology and Laboratory Medicine 2000:46-47). Although most of the sample is collected, unlike the Pap-test, only a small proportion of the cells are transferred onto the slide; less than 10% on a typical specimen. The remainder of the cells are left in the preservative fluid.

The ThinPrep pap test also comes at an increased cost. These include all of the costs of a conventional Pap screen in addition to ThinPrep specific costs. Pap screening costs include slides, spatulas, histotechnologist's time, Papanicolaou staining reagents, staining machine rental and operational costs, cover slips, cytotechnologist's screening time, pathologists time for review of abnormal slides, pathologists screening time if no cytotechnologist is available, and pathologists time cost for review of 10% of negative preparations as part of quality control protocol. In addition to these, ThinPrep pap test specific costs include (1) cost of vial with methanol based fluid preservative, approximately $10 (2) purchase of ThinPrep machine (2) purchase of ThinPrep machine reagents (4) and cost of training pathologist's and cytotechnologist's to interpret ThinPrep pap test slides. As a consequence, the major commercial laboratories in the United States charge up to $60 for the processing and screening of a ThinPrep pap test. This does not include the additional cost incurred should the slide have an abnormality that would require review by a pathologist.

An alternate FDA approved automated method of preparing cervical scrape specimens for analysis has been devised by Tripath Corporation via the Autocyte machine. As of 2003, only 7% of cervical scrape specimens in the United States were processed using the Autocyte machine. This is also a liquid based method of preparation in which the cervical scrape/broom brush devise is immediately submerged in an ethanol based preservative after tissue is obtained. The cellular material is dislodged into the preservative liquid. The cellular material is then centrifuged into a pellet. The cellular pellet is resuspended in concentrated form and the cells are then allowed to settle onto a slide. The end result is a monolayer of cells on a slide. The slide is then stained by the Papanicolaou method and examined by the cytotechnologist and/or pathologist. This method has not demonstrated any superiority over the ThinPrep pap test. It has all of the limitations of the ThinPrep pap test and also comes at an increased cost relative to the conventional pap smear test. More importantly it still does not solve the problem of having a cytotechnologist or pathologist select the abnormal specimen primarily, instead of some automated method.

In today's age of technology it does not make much sense that a screening method for cervical scrape specimens require the manual examination of such a specimen under the microscope for an abnormality. This is a very labor intensive and specific test. As mentioned earlier, review of a pap smear is the most labor intensive, least paid, and most litigious specimen that is examined under the microscope. Screening tests are designed for their high sensitivity, which usually comes at the cost of reduced specificity. That is, when designing a screening test one wants to make sure that it identifies all or as many of the true positives as possible even at the cost of having a number of false positives. Take for instance the examination of a patient's peripheral blood. This test is first examined by a machine, which gives a complete blood count with an automated differential count of cell types in the blood. If there is a population of cells outside the reference range or if there are a significant number of atypical cell forms identified, the machine will flag the case for review. The pathologist would then review the peripheral smear of the patient and provide a more specific analysis of all blood cell types, shapes, numbers, and quality. A specific differential or definitive diagnosis would then be generated. Such an automated screening test followed by either selective specific review by a pathologist or yet another automated specific test is what is required for the examination of cervical scrape specimens.

Such a screening test, as with all screening tests, would need to meet certain criteria to justify its introduction into the clinical arena. Three of these are as follow: (1) excellent sensitivity, (2) a meaningful result; that is, if the test is positive it should generate a meaningful follow-up and (3) the benefits of screening should be worth the cost.

Image analysis technology has been researched in an attempt to fully automate the screening of monolayer preparations of cervical scrapes. One such technology named Papnet still requires obligatory review of a certain number of fields selected by the computer which displays them onto a monitor for such. Another form of automated image analysis technology called AutoPap does not require an individual to look at the pap slide. Due to its diagnostic limitations however, this form of analysis is limited to rescreening of the 10% negative population selected for quality control. Neither of these technologies has reached clinical diagnostic acceptance. (1) The sensitivity is either too good or too poor giving (2) unmeaningful positives. (3) The cost is greater than all other described alternative technologies.

A proposed alternative approach would be to perform molecular diagnostic testing for HPV as a screening methodology for all cervical scrape specimens. According to a 1999 study published in the Journal of Pathology, HPV is present in virtually all cervical cancers, an estimated 99.7%. HPV is also detected in over 90% of CIN lesions. (Lorinez A T, Reid R, Jensen A B, et al.: Obstet Gynecol 1992; 79:328-337). According to the ASCUS LSIL Triage Study (ALTS) HPV testing was 96% sensitive for detecting CIN 3+vs. 85% for repeat ThinPrep pap test cytology on ASCUS. Additionally HPV testing confirms absence of disease with an average negative predictive value of 99%. (Salomon D, et al. Comparison of three management strategies for patients with atypical squamous cells of undetermined significance: Baseline results from a randomized trial. J Natl Cancer Inst. 2001; 93:293-9).

Currently HPV testing can be done by either in-situ hybridization or hybrid capture methodologies to identify specific sequences of the HPV genome. Although most cervical dysplasias (90%) and almost all cervical cancers (99.7%) are HPV positive the converse is not true. That is, a large percentage of women who are HPV positive have no identifiable lesion, nor do they ever go on to develop lesions. These are thought to be subclinical infections. Therefore implementation of this technology as a screening protocol would give rise to oversensitive and unmeaningful results that would be too expensive. In light of the results from the ALTS study; however, the Dygene HPV hybrid capture test has been FDA approved as an adjunct reflex test to the ThinPrep pap test for cervical cancer screening and detection once an ASCUS diagnosis has been rendered.

Unlike the Hybrid capture HPV DNA test, pap smears which have been on the market for several decades, are currently offered at levels equal to production costs are not likely to become less expensive. In fact, the introduction of fluid based methods of collection while making it possible to perform HPV testing from such have drastically increased the cost of the pap smear. The reason for the increased cost is that a monolayer slide preparation is prepared from the cells suspended in the fluid which are interpreted visually by a cytologist and/or pathologist. Since the Ventana method of performing insitu hybridization on a slide also requires a cellular monolayer preparation and cytologist/pathologist interpretation, it is also unlikely that this approach would have the capability of lowering costs to any significant degree.

The proposed method of automated cervical cytology screening would offer advantages over both current morphology and HPV testing methodologies. Automation of both modalities of detection would eliminate the pathologist/cytologist from the primary screening of cervical cytology specimens. The performance of both methods of detection directly on the fluid suspended cells would eliminate the cost incurred by slides, filters, and machinery used for current monolayer preparations (Thinprep). In addition to the elimination of these costs, the application of automated morphology and HPV DNA testing directly from the cell suspension also offers the potential for drastic cost reduction with increased volume.

In summary, all current cervical/endocervical gynecologic accepted screening diagnostic methods require staining of scraped cells by the Papanicolaou method and eventual examination of these cells by either a cytotechnologist or pathologist. In addition to the limitations described, this comes at an enormous financial burden. There are approximately 50 million pap tests performed in the United States annually of these 3.5 million women (7.0%) are diagnosed with an ASCUS result. This together with the addition of reactive and dysplastic categories accounts for approximately 10% of all pap tests, which have to be screened by a cytotechnologist and then reviewed by a pathologist. Accordingly, a successful automated pap screening machine would eliminate human screening of 45 million pap smears per year. A hypothetical cost of $30 per test for such a methodology instead of the $60 cost for a ThinPrep pap test would represent a $1.35 billion yearly savings. Aside from getting women to doctors for regular pap tests, successful automated screening of cervical/endocervical scrape specimens would be the most significant advance in cervical cancer screening since the development of the pap smear test.

The solution is the application of a totally different automated technology for the preparation and screening of a cervical/endocervical scrape specimens. This device would (1) test cells directly from a fluid medium, (2) analyze all of the fluid, (3) eliminate human evaluation of normal specimens, (4) have good sensitivity, (5) have a meaningful positive and (6) be cost effective.

### SUMMARY OF THE INVENTION

In accordance with the invention, systems and methods for automated screening of cervical/endocervical scrape specimens for premalignant/dysplastic lesions are provided. The specimen is collected in a fluid based medium and the suspended cells are analyzed by means of flow cytometric technology. Direct analysis of the cells in a fluid based medium with minimal manipulation would allow for analysis of such cells in an environment closer to in vivo than any other method currently available. This would also obviate the need to make a slide preparation of the sample to be analyzed, a prerequisite of any other currently available method. The elimination of slide preparation translates to reduction of time, morphologic artifact, and cost.

Screening of cervical/endocervical scrape specimens by flow cytometry allows for complete automation of the process. In one embodiment of the invention, analyzed samples can be recollected for further morphologic visual analysis or molecular DNA analysis. Alternately, residual unprocessed fluid can be subjected to such analysis. In another embodiment of the invention, specific atypical cell populations within a sample can be sorted and collected for further visual morphologic or molecular DNA analysis (FIG. 1).

Data relating to the cellular viability, number of cells examined, and relative percentage of each population type will be displayed in both a tabulated form (FIG. 4) and 3 dimensional graphical form (FIG. 5). Cell populations with significant atypia will be automatically flagged as abnormal.

In yet another embodiment of the invention, flow cytometric technology will be used in conjunction with DNA fluorescent in-situ hybridization (FISH) technology to detect human papilloma virus (HPV) infected cell populations. Identification of such populations will be correlated with morphologic flow cytometric information. All the information can be displayed simultaneously in 3 dimensional graphical form (FIG. 5). Alternately, specific atypical populations can be gated and analyzed for positivity of both high risk and low risk DNA probes (FIG. 3). As before, cellular populations that are HPV DNA positive with morphologic atypia can be selectively sorted for morphologic confirmation (FIG. 1).

The invention offers the following advantages over any other existing technology for cytological screening of cervical/endocervical scrape specimens: (1) total automation (2) elimination of glass slide preparation (3) 3 dimensional cellular analysis (4) 3 dimensional cellular population graphical representation (5) cell population type quantitation (6) ability to sort and collect specific cellular populations for further analysis (7) Use of FISH technology for detection of HPV infected cell populations (8) ability to truly examine all of the cells in the fluid bases sample (9) least labor intensive method available (10) potentially cheapest method available (11) automated correlation of morphology and DNA molecular diagnostic information (12) automated interpretation of such results.

Accordingly, it is an object of the present invention to provide an improved system for cytological screening.

It is a further object of the present invention to provide an improved method for cytological screening.

It is yet another object of the present invention to provide improved automated screening of cervical/endocervical scrape specimens for premalignant/dysplastic lesions.

The subject matter of the present invention is particularly pointed out and distinctly claimed in the concluding portion of this specification. However, both the organization and method of operation, together with further advantages and objects thereof, may best be understood by reference to the following description taken in connection with accompanying drawings wherein like reference characters refer to like elements.

### BRIEF DESCRIPTION OF THE DRAWINGS

FIG. 1 is a schematic view of a system in accordance with the invention;

FIG. 2 is a plot showing different cell nuclear morphology types;

FIG. 3 shows dot plot graphs representing detected light signals;

FIG. 4 is a representative chart showing cell type count classifications; and

FIG. 5 is a #D representation of different cervical squamous cell populations with possible human pailloma virus infection patterns.

### DETAILED DESCRIPTION

According to a preferred embodiment of the present invention, automated systems and methods are provided. A cervical/endocervical scrape specimen is introduced into a liquid based transport medium. The transport medium should have several properties. (1) It may consist of either a methanol or ethanol fixative or perhaps another fixative to retain cellular morphology and allow cells to retain the ability to be analyzed by molecular methods explained later. (2) An isotonic osmolarity medium to maintain cellular volumetric integrity (3) the medium would need a mucolytic agent for endocervical mucous. (4) A blood lysing agent such as ammonium chloride or acidic acid (5) a cellular preservative (6) a cellular ion agent to break up groups of cervical and endocervical cells so that they can be analysized individually (7) an anticoagulant such as heparin sodium (8) a possible stain to allow for cellular detection. (9) A nuclear stain for detection of nuclear size and nuclear surface irregularities. (10) An agitation step may be provided either previous to the introduction of the specimen to the automated device or as an early-automated step of the device itself. The cervical scrape/endocervical specimen is now ready to be analyzed by the automated device as single cells devoid of mucin or blood.

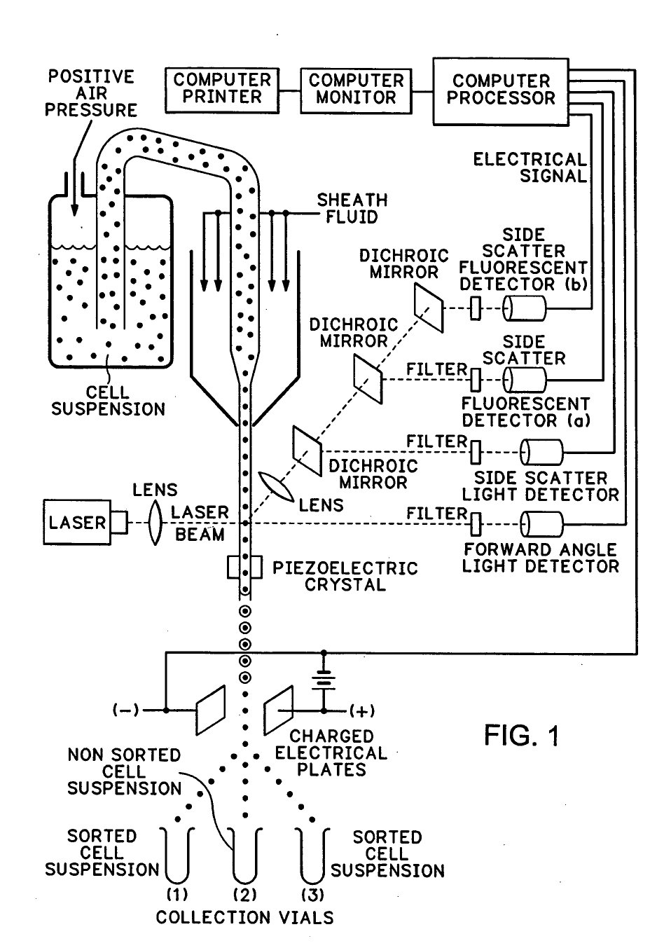

The device first agitates the specimen gently to disrupt residual cellular groups. As mentioned earlier this may also be done manually prior to the introduction of the specimen to the device. The device then suctions the fluid based specimen from the vial through negative pressure. The method of specimen analysis is through flow cytometry technology. Flow cytometry is a process by which cells pass singly in a fluid stream. The exact methods of achieving this may vary. It may be achieved by suspending the cells in isotonic fluid medium and introducing it into a nozzle shaped chamber with a small exit diameter. (FIG. 1). Isotonic fluid is also introduced into a sheath chamber surrounding the sample chamber in order to create a laminar flow. The differential pressure under which the sample and the sheath fluid are forced into the nozzle results in concentrating the cells in the center of the fluid stream in which the pass singly.

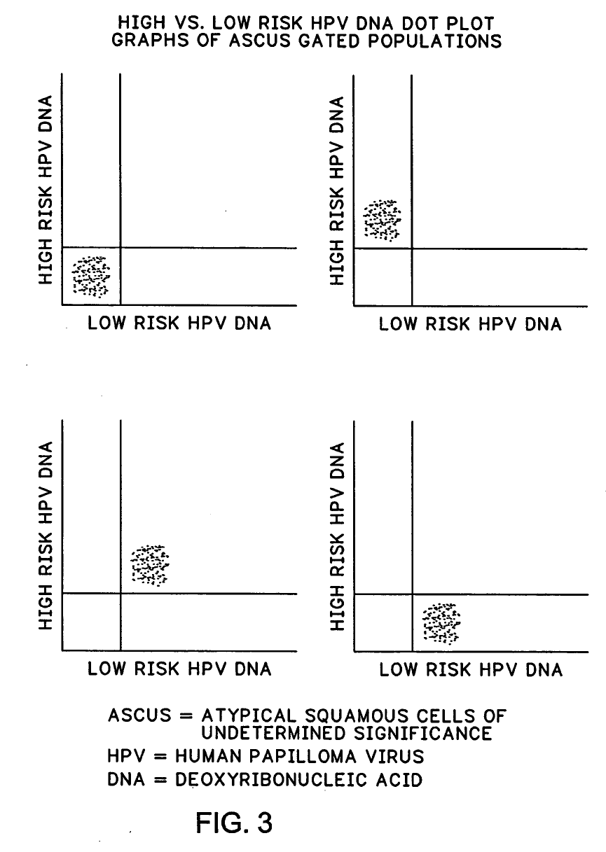

The second component involves the detection of the cells and the properties of such as they pass singly. The three principle features used to establish morphology are (1) nuclear size (2) nuclear surface irregularity and (3) cell size. The nuclear to cytoplasmic ratio is a 4^{th} calculated parameter. This is accomplished by passing a beam of light (typically a laser beam) through the laminar cell flow. Photons are separated and collected by both forward and side light scatter detectors. (FIG. 1). The forward detector is placed directly in line with the direction of the light beam on the opposite side of the laminar flow or cells being analyzed. The side light scatter detector is usually placed at a 90-degree angle to the laser beam but it may not necessarily be at this exact angle. Photo multiplier tubes then convert the detected light signal into a digital signal so that the data can be analyzed and finally be plotted into a dot plot graph or screen (FIG. 3)

Forward light scatter gives an indication of the size while side light scatter reflects nuclear complexity and/or cytoplasmic granularity. In order to differentiate between nuclear size and cytoplasmic size several methods may be employed. (1) The cell size can be measured by a change in electrical resistance of a restrictive flow channel due to the presence of the cell in the channel. This is an old technique used by old coulter counters, see, for example, U.S. Pat. No. 3,497,690. This method can be combined with a nuclear stain. By using such a stain the degree of light transmission detected by the forward photomultiplier detector reflects the nuclear size. (2) Specific nuclear DNA and cytoplasmic stains can be used. In this way the differential light transmission detected in the forward photomultiplier tubes can differentiate between nuclear size and cell size. See, U.S. Pat. No. 3,657,537. (3) the cell size can be measured by allowing cells in liquid to flow through a predetermined volume as a steady electromagnetic field is established within the testing volume. A change in the electromagnetic field caused by the passage of a cell is measured. This measures the cell size. Again this can be combined with a nuclear stain such as a DNA stain. As before the degree of transmitted light detected by the forward photomultiplier tube correlates with nuclear size. See, for example, U.S. Pat. No. 3,770,349. With any of these methods the N/C ratio would be a calculated value.

Since cervical and endocervical cells have very little cytoplasmic granularity the detected light by the orthogonal photomultiplier tube will predominantly represent nuclear surface irregularity. Traditionally the orthogonal photomultiplier tube has been used by flow cytometers to represent cytoplasmic granularity. Its use for detection of nuclear surface or perhaps internal nuclear irregularities would require calibration and correlation between morphologically normal and abnormal cells. Also, variation index between cells may be used in detecting abnormal nuclear features. Occasionally squamous cervical epithelial cells do have so called keratohyalin cytoplsmic granules observed with a Pap stain. Such granules are said to be a soft criteria for dysplasia. The degree to which unstained samples with such granules would contribute to the complexity parameter in unknown. But since both nuclear surface irregularities and the presence of keratohyalin granules are features of dysplasia their contribution to cellular complexity is complementary. A stain can be used that will stain both the nucleus and cytoplasmic granules.

The criteria used to identify squamous intraepithelial lesions and glandular lesions may include but are not limited to the following: nuclear size, nuclear membrane complexity, cell size, Nuclear/cytoplasmic ratio, nuclear hyperchromasia, and nuclear hypochromasia. The following parameters may be employed, for example: (1) placement and number of the scatter detectors to optimally differentiate squamous intraepithelial lesional cells and dysplastic glandular cells from normal cells (2) threshold of complexity for lesional cells (3) threshold of nuclear size for lesional cells (4) threshold N/C ratio for lesional cells optimizing laser wavelength for analyzing squamous cells and glandular cells (5) optimizing laser wavelength to distinguish cell size from nuclear size and thus obtain an N/C ratio (6) establish hyperchromasia and hypochromasia thresholds (7) optimize laser wavelength and/or intensity to variably distinguish nuclear chromasia (8) optimize cell suspension components and concentrations.

The detection of cells infected with human papilloma virus (HPV), distinction between different types (high risk and low risk HPV) of HPVS, and the correlation of HPV infected cells and cellular morphology can be accomplished by automated fluorescent in-situ hybridization (FISH). The process involves six steps. (1) permeabilization (2) enzyme digestion (3) denaturation (4) hybridization (5) stringency washes and (6) detection.

(1) Permeabilization. Unlike the majority of antigens detected on hematopoietic cells by flow cytometry, in-situ hybridization designed probes will have to be detected in the nuclei of cells. This procedure would require the need to permeabilize the cell membrane. The permeabilization step provides the mechanism by which the fluorochrome-conjugated probe can enter the cell so that nuclear binding by the probe can take place.

(2) Enzyme digestion. DNA and RNA strands are relatively unaffected by fluid fixatives. Their nucleic acid sequences molecules are however protected by cross-linked proteins. These proteins would prevent a nucleic acid probe from gaining access the cell DNA. In order to overcome this problem the liquid based cells undergo digestion by a protean specific enzyme or protease.

(3) Denaturation DNA strands occur as two chains of individual nucleic acids which are complementary to each other and twisted into a double helix. The double helix is held together by hydrogen bonds. In order for a DNA or RNA probe to bind to the cells DNA the cells double strands have to be separated from each other. The method by which the DNA hydrogen bonds are broken so that the individual strands of DNA unfold is called denaturation. This can be accomplished either by heat or chemical methods. Note, the DNA or RNA probes do not require denaturation because they are introduced to the cells as single strands.

(4) Hybridization. Now that the individual strands of DNA are exposed, a DNA or RNA labeled probe is free to bind or hybridize to a complementary sequence specific segment of cellular DNA. The hydrogen bonds between the probe and the cells DNA are however weak and the probe is in constant competition with the cells complementary DNA. In order to solidify the probes hydrogen bonds to the cells DNA the temperature is either decreased or the chemical conditions for denaturation are neutralized.

These four steps can be performed in a fluid state and automated by sequentially adding the appropriate enzyme or chemical. Each enzyme or chemical used can also be neutralized by the addition of a neutralizing agent before proceeding to the next step.

(5) Stringency washes. These are washes that are performed after the hybridization step. The purpose of the washes is to remove any probe that has not bound to cellular DNA. The washes also serve to remove probe that has bound to undesired portions of DNA nonspecifically. The binding of probe to such undesired segments of DNA is less stable than the more specific areas that the probe was designed to bind. The stringency of the washes can therefore be manipulated such that the probe is removed from the undesired nonspecific portions of DNA and not the specifically desired segments. The stringency of the washes of course requires certain research and development. Another area is the identification of a method of performing these washes in a fluid based specimen altogether. Usually a labeled probe is attached to a portion of DNA, RNA, or protein. These may be intracellular or not. However the component to which the probe is bound is usually attached to a solid state. In this way the bound probe is kept from being washed away. The cells in a fluid based specimen may have to temporarily be attached to a solid state for the washes to be effective. Otherwise a differential gradient may be devised such that the wash with unbound probe will wash ahead of the intracellular bound probe. Other alternatives may be devised.

(6) Detection. Many methods of nonisotopic labeling and detection have been developed. In some applications, nucleic acids are directly linked to a signal generating compound, usually a fluorochrome but occasionally an enzyme. Nucleic acids are indirectly detected in a multistep fashion. Biotin, a commonly used affinity label was the first label introduced into nucleic acids (Lager, 1981). Biotin itself generates no signal, but is detected by high-affinity interaction with an avidin or streptavidin molecule that is in turn is complexed or conjugated to a signal-generating enzyme or fluorochrome. Many other functional groups have been developed as nonisotopic labels such as bromodeoxyuridine, digoxigenin, and sulfone. Detection in these cases is achieved with high-affinity antibodies directed against the functional group. These antibodies are usually directly linked to a signal generating enzyme or fluorochrome, functioning like a labeled secondary antibody in an immunohistochemistry reaction. Biotin may also be detected with an antibiotin antibody rather than an avidin or streptavidin molecule. For the purpose of detection by flow cytometry the use of a fluorochrome would likely be the best signal-generating compound. The fluorochromes typically used in clinical flow cytometry have specific distinct excitation and emission spectra. The argon laser is the most commonly used light source. It produces a 488-nm excitation wavelength, which is able to excite many different fluorochromes. Two of the most widely used fluorochromes that can be excited by this wavelength are fluorescein isothiocyanate (FITC) and phycoerythrin (PE). These fluorochromes are commonly used simultaneously because they can both be excited by the same light source yet have different spectra of light emission. It is their difference in light emission that allows them to be detected as separate signals and wavelength groups. This feature would be most convenient in the detection of human papilloma virus (HPV) from cervical/endocervical scrape specimens. Clinically significant forms of these viruses are classified into high risk and low risk types. Therefore if all of the high risk probes for HPV are labeled with FITC and the low risk probes for HPV are labeled with PE, for instance, then detection of cells for both types could be done simultaneously. Texas red (TR) and Cynanine 5 (Cy5) are two other fluorochromes that can be coupled with PE to produce tandem conjugate fluorochromes and in turn increase the number of probe types that can be detected simultaneously. The actual detection of the signal is accomplished by placing a fluorescence detector in the area of the side scatter detector (other location may be possible). In fact, newer flow cytometers contain two lasers or four detectors that can simultaneously excite and detect four or more fluorochromes (four-color flow cytometry) on a single cell.

In addition to having the capability of distinguishing different populations of cervical/endocervical cells by morphologic parameters and cytofluorescence positivity, the system/method also has the capability of sorting cell populations by such parameters. Preparative cell sorting process involves the physical separation of a subpopulation of cells from the main population. This capability evolved from ink-jet printer technology and is elegantly simple in concept. In cell sorters, the flow chamber is seated in a piezoelectric crystal that vibrates in response to a coupled acoustical transducer. The vibration of the crystal is imparted to the flow chamber and causes the stream to form nodes and eventually to break up into droplets. The droplet formation should occur downstream so it does not disturb the laser interrogation point. To engage the sorting mechanism, a logic is established with the computer system that instructs it to look for a cell which satisfies a given sort criteria. These criteria may include gates on any property of any measurable parameter. At the point of laser interrogation, the computer determines whether or not the cell satisfies the sort criteria. If it does, the instrument places an electrical charge of a given polarity on the entire stream. When the droplet containing the cell of interest breaks away from the stream, the entire stream is discharged. The only charged particle in the sample flow is now the droplet containing the specific cell. As the droplet moves downstream at a speed of 10 meters per second, it enters an electrostatic field created by 2 charge plates; one plate carries a negative charge, the other a positive charge. The charged droplet is attracted to the plate of the opposite charge and is deflected from the mainstream. It is then a simple matter to place a collecting vessel in the path of the sorted droplets. In commercial instruments, 2 different populations may be simultaneously sorted. Cells collected in this manner are viable, may be maintained sterile, and may be bulk sorted or index sorted by automatically sorting single cells into the wells of microtiter plates.

Some manufacturers have introduced clinical flow cytometers with low speed sorting capabilities. One example of a low speed clinical sorter is the FACSort (Becton Dickinson Immunocytometry Systems, San Jose, Calif.), which uses a mechanical means of sorting cells rather than the traditional Jet-in-air approach in which cells are sorted by electrostatic charges. The concept behind low-speed sorters is that they allow the pathologist to correlate morphology with phenotype by sorting small numbers of cells with specific characteristics for later analysis by cytospin or monolayer slide preparations. Additionally, sorting may be used as a preparative technique for molecular tests including immunohistochemistry, PCR, or in-situ hybridization. For cervical/endocervical testing, in-situ hybridization for high risk and low risk HPV viruses would be the test of choice.

The device therefore has three major functions (1) identify abnormal cervical/endocervical cell populations by morphology (2) identify abnormal cervical/endocervical cell populations by in-situ hybridization based fluorescence (3) sort identified abnormal cervical/endocervical cell populations.

The order in which these steps are performed may vary. In-situ hybridization may be performed first so that the machine may then simultaneously identify a morphologically abnormal population of cells that is also abnormally fluorescent. This population will then be sorted for subsequent slide preparation and microscopic correlation. It may however be logistically difficult to perform in-situ hybridization on the entire volume of sample fluid. If so a concentration step may have to be performed first. If this would not be possible and in-situ hybridization would require a lower fluid volume, then the order of the sequence would have to be changed. The reason is that one of the major advantages of flow cytometry based screening is that the entire volume of cervical/endocervical fluid may be processed as opposed to a minor fraction with the current fluid based screening methods. The sequence necessary to satisfy this potential problem is as follows. First the flow cytometer will identify a morphologically abnormal population of cells. These cells are then sorted. This sorted cell population then undergoes in-situ hybridization and a subsequent identification of abnormally fluorescent cells. These can then be additionally sorted and used for slide preparations. Alternately, initial morphologically abnormal sorted cells can be divided into two aliquots, one for in-situ hybridization and one for microscopic examination. Note, the in-situ hybridization can be done either with or without fluorescence. If done with fluorescence the machine can analyze it. If done without fluorescence a biotin-avidin antibody based approach can identify positive cells with light microscopy.

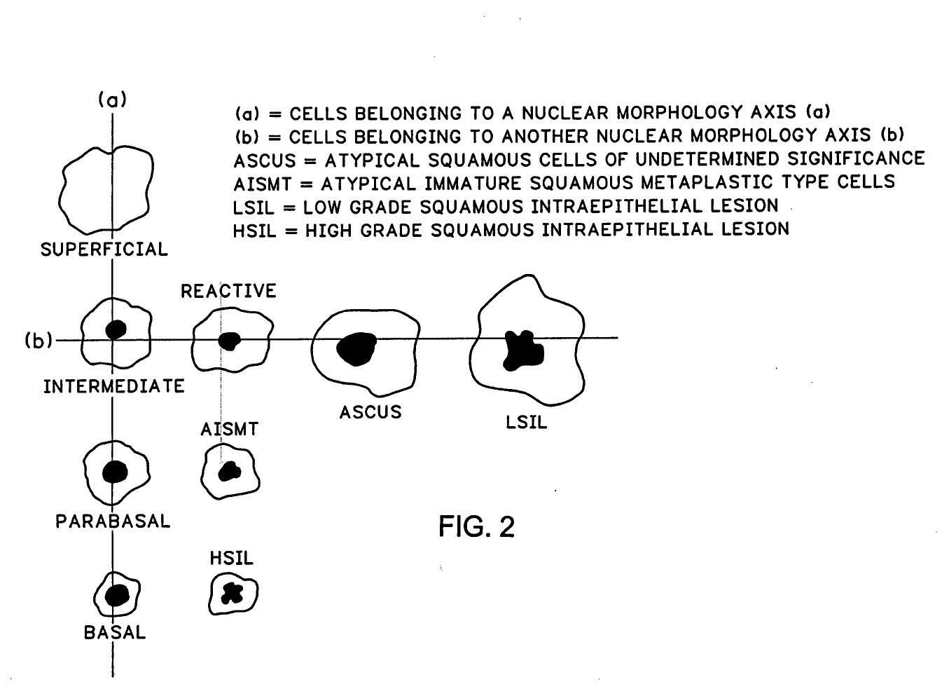

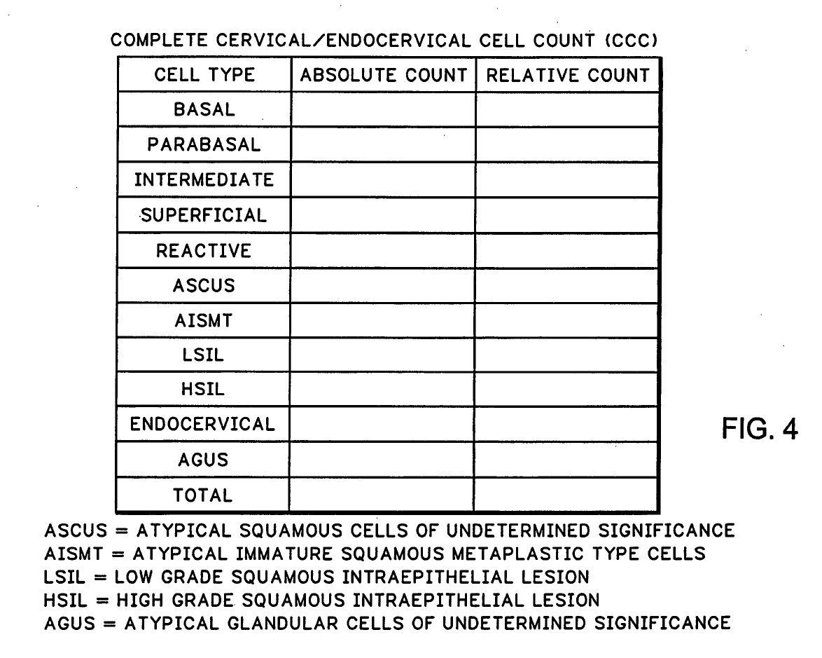

The final phase of the invention is software logic that simultaneously integrates all of the detected parameters. This is accomplished by first quantifying each of the detected parameters for each of the cells examined. The parameters of interest are (1) cell volume (2) nuclear volume (3) nuclear to cytoplasmic (N/C) volume ratio. This is not a directly measured value but rather a calculated value resulting from dividing the nuclear volume by the cell volume. (4) Nuclear irregularity (5) fluorescence of nuclear probes for both high risk and low risk HPV virus. The degree of fluorescence can also be measured and may correlate to viral load. The degree of side scatter fluorescence may also reflect weather the HPV genome is episomal or integrated. The latter has an association with progression of disease and may have clinical significance. From the initial non fluorescent flow cytometric morphologic analysis a quantitative report can be generated depicting (1) the total number of cells examined (2) adequacy of specimen as determined by appropriate number of cervical and endocervical cells (3) percentage of total cells comprised of individual population types detected as well as absolute number of cells in each population (FIGS. 2 and 4).

Thresholds of normality are established for all of the above parameters. Such thresholds should be correlated with current morphologic criteria for the detection of squamous intraepithelial lesions. The standard for such criteria would be those used by the latest revised Bethesda System. A logic displays each of the cells parameters in plot format. Any combination of the 5 parameters can be plotted on two dimensional Cartesian coordinate systems to segregate populations visually. However, the most useful visual representation would be a three dimensional plotting system. This would allow for a simultaneous visual representation of all of the populations of cells by simultaneously representing all of the measured parameters. Individual thresholds of normality previously established could then be superimposed on this three dimensional construct to identify abnormal cellular populations.

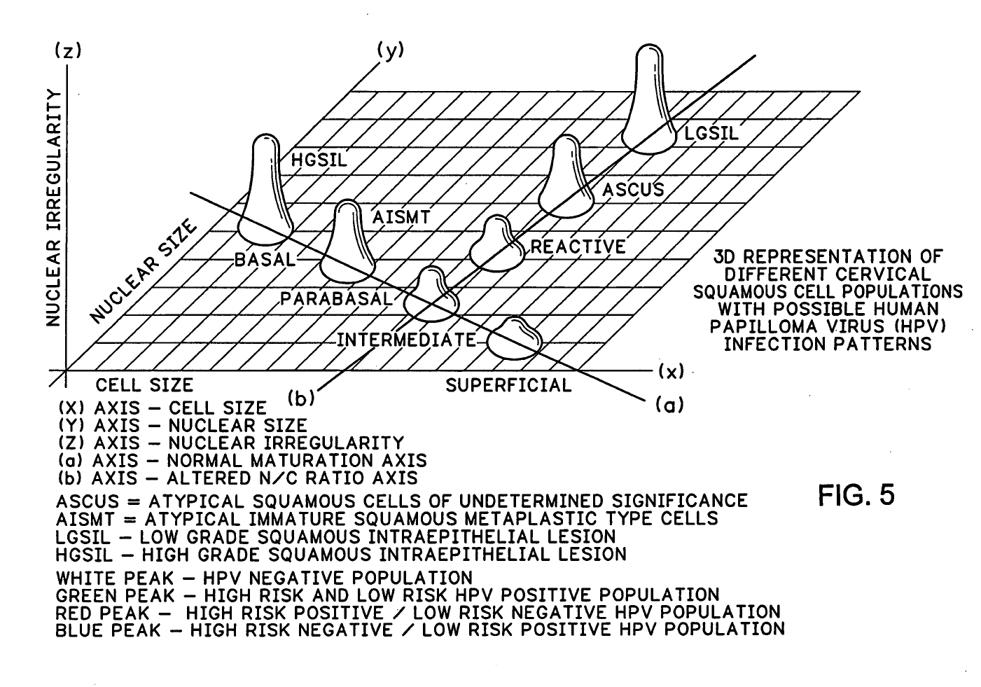

Such a visual three dimensional representation can be constructed as follows (FIG. 5): A horizontal x axis represents cellular volume. A second horizontal axis y represents nuclear volume. And a vertical z axis represents nuclear irregularity. The major populations of epithelial cells encountered in cervical/endocervical scrapes occupy corresponding regions in this three dimensional coordinate system. For completeness the features of these cells will be described (FIG. 5) in conjunction to their corresponding regions. Ectocervical cells progressively mature from basal cells to parabasal cells to intermediate cells to superficial cells. As these cells mature the nuclear volume progressively decreases, cellular volume progressively increases and consequently the nuclear to cytoplasmic ratio is reduced. There is no significant nuclear irregularity expected in the normal ectocervical maturation from basal to superficial cells. These progressive morphologic changes would be expected to appear along line (a) in the xy plane of our above described coordinate system. Basal cells with the smallest volume would appear adjacent to the y axis at an area reflective of their nuclear volume. Superficial cells, on the other hand, with the smallest nuclear volume would be expected to appear adjacent to the x axis at an area reflective of their cellular volume. Parabasal and intermediate squamous ectocervical cells would appear along line (a) in corresponding positions between the basal and superficial cellular populations.

Ectocervical squamous cells displaying cytologic features of high grade dysplasia are termed High Grade Squamous Intraepithelial Lesional (HSIL) cells. These cells basically have the same volume and nuclear size as parabasal cells (FIG. 2). Consequently they are expected to appear in the same position along the xy plane as parabasal cells. HSIL cells, however, have distinct and significant nuclear surface and internal irregularities not present in basal cells. These nuclear features are reflected as a z oriented peak extending from the xy plane parabasal region (FIG. 5).

Similarly so called Atypical Immature Squamous Metaplastic Type (AISMT) cells have similar volume and nuclear size to parabasal cells. Again the cytologic difference lies in nuclear irregularity. The nuclear irregularities however are not as marked as those in HSIL cells; otherwise they would be classified as such. Note, these cells may actually be dysplastic HSIL) but the criteria fall short of classifying them as such. These cells would correspondingly appear on the parabasal region along the xy plane. Additionally the nuclear irregularities would be expressed a peak along the z axis that is shorter in stature than that seen for HSIL cells (FIG. 5).

The intermediate cell is the cell to which all other squamous ectocervical cells are compared to when considering their type, reactivity status, or squamous intraepithelial lesional (SIL) status. It is therefore not surprising that this cell type occupies a central position in both our cytologic diagrammatic chart as well as in our three dimensional coordinate system (FIGS. 2 and 5). Cells with progressively increasing nuclear volume, nuclear irregularity, and cellular volume include reactive squamous cells, atypical squamous cells of undetermined significance (ASCUS) and squamous cells displaying low grade dysplastic cytologic features called Low Grade Squamous Intraepithelial lesional (LSIL) cells. These cells occupy corresponding regions along line (b) on the xy plane. Note, reactive cells have slightly larger nuclear size and greater nuclear irregularities compared to intermediate cells. ASCUS cells are defined as cells whose cytologic features greater than reactive cells yet fall short of LSIL cells.

Unlike nuclear volume and cellular volume, nuclear irregularity should always be considered an abnormal finding. Only the degree of the nuclear irregularity peak in a given population of cells will determine weather it would be categorized as either an atypical or squamous intraepithelial lesional population. These thresholds are correlated with morphology and developed.

Unlike ectocervical cells, endocervical cells are glandular. These cells would have approximately the same volume and nuclear volume as an intermediate cell. The difference is in the tissue organization of these cells and their nuclear polarization. Therefore one would expect to find these cells in the vicinity of intermediate cells. Note as with squamous epithelial cells there should be no significant z peak reflecting nuclear irregularity. A minor peak may be indicative of reactive changes and taller peaks may represent an atypical change or dysplasia.

The light emitted by a fluorochrome that is bound to an HPV DNA probe will be detected and translated as a specific color in the logic system (FIG. 5). For example a cell population with positive nuclear staining for low risk HPV DNA probe may be displayed in blue (the AISMT entry in FIG. 5). A high risk positive nuclear staining population may be displayed in red (the HGSIL and ASCUS entries of FIG. 5). A population of cells with positive nuclear staining for both high risk and low risk probes may be displayed in green (e.g., the LGSIL entry of FIG. 5). Cellular populations devoid of HPV nuclear staining may be displayed in white (the REACTIVE, INTERMEDIATE and SUPERFICIAL items of FIG. 5).

Practically it may not be cost effective to subject all of the samples for HPV testing. 75-90% of the samples would be regarded as negative for dysplasia by non fluorescent flow cytometry alone. Many possible algorithms can be devised for populations that are regarded either atypical or lesional by non fluorescent cytometry alone. Some of these algorithms include the following:

1—Program the system to categorize all of the cell populations in normal or reactive xyz regions as negative. Populations in ASCUS or SIL xyz regions would be categorized as positive. Negative samples can be reported as such and the residual fluid discarded. In samples that are positive the entire fluid would be recollected and subjected to monolayer slide preparations for microscopic inspection. Microscopic lesional cells would be reported as HSIL or LSIL and the remaining fluid again discarded. The residual fluid from microscopic ASCUS cases can however be used for flow cytometric HPV in situ hybridization. HPV fluorescent populations in morphologically positive regions should be considered and reported lesional. Note, this system is able to correlate morphologically positive populations with HPV fluorescence.

2—Since the ASCUS category falls morphologically between reactive and SIL categories the programming threshold for ASCUS could be lowered to include a greater number of populations that would have otherwise been categorized as reactive at one end and SIL at the other. This would in effect raise the threshold for both reactive and SIL populations. The specificity for both reactive and SIL populations are raised in this manner. This can be done to the point where the negative predictive value (NPD) for SIL is 98% if only reactive or normal cellular populations are identified. Conversely the positive predictive value for a lesion would also be about 98% if a SIL population is identified. Under this system both negative (reactive or normal) and positive (SIL) cases can be reported as such with no further testing and the residual fluid can be discarded. There will however be a larger number of cases categorized as a typical. Again the residual fluid from this category can be used for microscopic examination. Some of these will be categorized as reactive and others as SIL. Again these can now be signed out as such and the residual fluid discarded. A number of cases will remain in the atypical category and signed out as ASCUS. Residual fluid can then be subjected to flow cytometric FISH analysis for HPV. As before HPV fluorescent populations in atypical regions should be considered lesional and reported as such. Additionally a comment can be added as to whether the region is suggestive of LSIL or HSIL. Non fluorescent cases can be reported as negative.

3—In both of the above examples the fluid residual for microscopic examination can be use subsequently for either of the current existing methods of HPV detection. That is monolayer slide insitu hybridization or hybrid capture. These options may be desirable for flowcytometric machines that do not have HPV fluorescent detection modules. Cost would be the major issue in this decision.

4—Another option is for the flow cytometer to have a cell sorter. A cell sorter would allow the flow cytometric atypical populations to be physically separated from all other populations. When the pathologist then makes a slide from such a preparation he/she would theoretically be looking at a pure population of atypical cells. This would increase the sensitivity for the microscopic detection of lesional cells dramatically and therefore eliminating the need for subsequent HPV testing in many cases.

5—The fully implemented system has (a) conventional nonfluorescent flow cytometric detection of squamous and endocervical cell populations (b) at least 2 color fluorescent detection capability for HPV. (c) Correlation software between fluorescent and appropriate morphologic populations (d) cell sorting capability. The combination of modules used, however, would depend on the patient population, type of practice, cost of testing, and acceptability of methodology and reimbursement for such.

6—One last module to be considered as part of this invention is the protocol previously described for liquid based insitu hybridization for HPV in squamous ectocervical and endocervical cells. This process can be done either manually or automated. If automated it may be incorporated as a module or a separate unit.

Accordingly, improved systems and methods for automated screening of cervical/endocervical malignant and premalignant epithelial lesions has been shown and described.

The system/method advantageously provides use of flow cytometry for the clinical evaluation and diagnosis of cervical/endocervical scrape specimens. Further, it provides automated direct analyzing and diagnosing fluid based cervical scrape specimen without need of slide preparation. Automated qualitative and quantitative identification of cervical scrape populations directly from collection fluid sample are possible with the invention as well as quantitation of total number of cells in cervical scrape specimen and determination of adequacy depending on number of ectocervical and endocervical cells detected.

The system can provide initial morphologic quantitative reports with adequacy, total number of cells examined, percentage of total number of cells comprised by individual population types detected, and absolute number of cell types expressed as a cervical cell count (CCC). The system/method further provides ability to process the entire fluid based cervical scrape specimen for clinical analysis and diagnosis and the ability to recollect analyzed fluid bases specimen for further analysis.

Still further, performance of fluid based in-situ hybridization for subsequent flow cytometric analysis for clinical detection and diagnosis of cervical scrape specimens is possible. Use of fluorescent in-situ hybridization for the detection of HPV positive cellular populations from cervical scrape specimens is accomplished with the invention.

Another aspect of the invention is use of flow cytometric technology for the detection of HPV FISH positive cellular populations from cervical scrape fluid specimens and detection of nuclear surface and internal irregularities by flow cytometry from fluid based cervical scrape specimens. Computer processing is employed for correlating cell size, nuclear size, nuclear irregularity, and cytoplasmic granularity as detected by flow cytometry for the diagnosis of cervical scrape specimens and for correlating FISH positive HPV cellular populations with morphologic features of those populations including cell size, nuclear size, nuclear irregularities, and cytoplasmic granularity as detected by flow cytometry.

The system and method provides the concept of sorting a population of cervical/endocervical cells based on morphologic and/or fluorescent in situ hybridization characteristics and the use of cell sorting technology for isolation of specific populations from cervical scrape specimens for further microscopic testing or molecular testing including immunohistochemistry, PRC, in situ hybridization, or fluorescent in situ hybridization. Still further, the use of cell sorting technology in cervical scrape specimen for further testing including flow cytometric FISH or subsequent monolayer slide preparations is enabled.

Clinical analysis and diagnosis of fluid based cervical scrape specimens that have non fluorescent flow cytometric analysis of the sample as the first mode of analysis may be employed. Also, use of a nuclear stain for the purpose of detecting nuclear irregularities by flow cytometry can be used. The system/method can combine detecting both nuclear volume and cell volume in flow cytometer.

The system and method use flow cytometry technology for the calculation of nuclear cytoplasmic ratio in cervical scrape specimens and generation of 3 dimensional graphical display of cell populations simultaneously representing cell volume, nuclear volume, nuclear irregularity, and high/low risk HPV status. Fluid based specimen collection and also fluid based analysis provides optimal environment for morphology analysis of cellular populations in their 3D natural state.

Another aspect provides correlation of degree of nuclear fluorescence with HPV probes with viral load. Automated discrimination between episomal and integrated nuclear pattern of fluorescence by flow cytometry is provided and accurate assessment of cervical scrape adequacy by giving cell count in the sample analyzed. The system and method can further provide automated maturation index by providing relative proportion of basal/parabasal, intermediate, and superficial cells.

While preferred embodiments of the present invention have been shown and described, it will be apparent to those skilled in the art that many changes and modifications may be made without departing from the invention in its broader aspects. The appended claims are therefore intended to cover all such changes and modifications as fall within the true spirit and scope of the invention.

What is claimed is:

1. A system for evaluation and diagnosis of cervical/endocervical scrape specimens, comprising:
flow cytometer for presenting specimens for analysis; and
an inspection device for inspecting and analyzing the presented specimens.

2. The system according to claim 1, wherein said inspection device comprises optical inspection and classification.

3. The system according to claim 1 wherein said inspection device employs fluorescent in-situ hybridization.

4. The system according to claim 3 wherein said system detects HPV positive cellular populations.

5. The system according to claim 1 wherein said system detects HPV FISH positive cellular populations.

6. The system according to claim 1, wherein said inspection device comprises:
a laser;
forward angle light detector;
side scatter light detector; and
at least one side scatter fluorescent detector.

7. The system according to claim 1, further comprising:
sorting apparatus for sorting specimen cells according to their analysis.

8. The system according to claim 7, wherein said sorting apparatus comprises electrical charge device for diverting a selected specimen cell to one of plural sorted cell suspension collection vials.

9. A method for evaluation and diagnosis of cervical/endocervical scrape specimens, comprising:
presenting specimens cells for analysis substantially discretely; and
inspecting and analyzing the presented specimens.

10. The method according to claim 9, wherein said presenting is accomplished by use of flow cytometry

11. The method according to claim 9, wherein said inspection comprises optical inspection

12. The method according to claim 9, further comprising the step of sorting said specimens based on inspection.

13. The method according to claim 9, further comprising generating a 3 dimensional graphical display of inspection analysis results.

14. The method according to claim 13, wherein said graphical display represents one or more of cell volume, nuclear volume, nuclear irregularity and high/low risk HPV status.

15. The method according to claim 9, wherein said presenting comprises employing flow cytometry.

16. The method according to claim 9, wherein said inspecting and analyzing comprises correlation of degree of nuclear fluorescence with HPV probes with viral load.

16. The method according to claim 9, wherein said inspecting and analyzing comprises automated discrimination between episomal and integrated nuclear pattern of fluorescence.

17. The method according to claim 9, wherein said inspecting and analyzing comprises providing assessment of cervical scrap adequacy.

18. The method according to claim 17, wherein said providing assessment comprises providing cell counts of the sample analyzed.

19. The method according to claim 9, wherein said inspecting and analyzing comprises providing relative proportion of basal/parabasal, intermediate and superficial cells.
